(19) United States
(12) Patent Application Publication
(10) Pub. No.: US 2005/0101535 A1
(43) Pub. Date: May 12, 2005

(54) Use of a synthetic alpha-melanocyte stimulating hormone agonist to decrease steroid induced weight gain

(76) Inventors: David H. Rosenstein, Las Vegas, NV (US); Michael A. Nasiak, Las Vegas, NV (US)

Correspondence Address: GREENLEE WINNER AND SULLIVAN P C, 4875 PEARL EAST CIRCLE, SUITE 200, BOULDER, CO, 80301, US

(21) Appl. No.: 10/841,961

(22) Filed: May 6, 2004

Related U.S. Application Data
(60) Provisional application No. 60/468,724, filed on May 6, 2003.

(51) Int. Cl. A61K 38/22

(52) U.S. Cl. 514/12

(57) ABSTRACT

People who are on chronic steroids are known to have depressed levels of ACTH and thus should also have decreased levels of α-MSH. Recent data show that people with altered Melanocortin-4 (MC4) receptors (receptor for α-MSH) have strongly associated rates (100%) of Binge Eating Disorder diagnosable of DSM IV criteria. All patients who are on steroids and have low levels of ACTH, and thus low levels of α-MSH, could appear phenotypically identical to those with altered Melanocortin-4 receptors (MC4R). Patients with iatrogenically induced α-MSH deficiencies (including patients on chronic steroid therapy) could be expected to respond to exogenous α-MSH or MC4R agonist supplementation. Exogenous α-MSH or MC4R agonist treatment should also decrease serum leptin levels; therefore, the present invention provides a method of decreasing steroid induced weight gain by employing α-MSH agonists.

### CROSS REFERENCE TO RELATED APPLICATIONS

This application claims benefit of U.S. Provisional Application No. 60/468,724 filed May 6, 2003, which is incorporated herein in its entirety, by reference.

### BACKGROUND OF THE INVENTION

The present invention relates to a method of regulating body weight in a subject by administering an alpha-melanocyte stimulating hormone (α-MSH) agonist, particularly, a method of decreasing steroid induced weight gain in those subjects who are on chronic steroid therapy by using a synthetic α-MSH agonist.

It has long been known that chronic steroid use can decrease corticotrophin releasing hormone (CRH) release from the hypothalamus and adrenocorticotropic hormone (ACTH) release from the pituitary in humans (FIG. 1). Pro-opiomelanocortin (POMC), a precursor peptide, is cleaved into a variety of peptides including β-lipotropin and ACTH. β-Lipotropin is further cleaved into γ-LPH and β-endorphin, and ACTH is further cleaved into corticotropin-like intermediate lobe peptide (CLIP) and α-melanocyte stimulating hormone (α-MSH) (FIG. 2). As α-MSH is a direct cleavage product of POMC, α-MSH levels would be expected to be reduced when POMC production is suppressed.

The hypothalamus has a MC4R receptor for α-MSH that mediates many of its physiological and endocrinologic effects [Farooqi, I S et al. (2003) New Eng. J. of Med. 348:1085-1095; Branson, R. et al. (2003) New Eng. J. of Med. 348:1096-1103; Hinney, A. (1999) J. Clin. Endocrinol. Metab. 84(4):1483-1486; List and Habener (2003) New Eng. J. of Med. 348:1160-1163]. Abrupt steroid discontinuation is dangerous, due in part to suppression of the hypothalamic/pituitary axis. Therefore, serum α-MSH levels would be expected to be reduced in this same patient cohort. It has been shown that leptin, a hormone released by fat cells, is involved with obesity. Patients with defective leptin genes are overweight and treating these patients with recombinant leptin results in a consistent decline in their weight [Lustig, RH (1 Sep. 2001) Endocrinol. Metab. Clin. N. America 30(3):765-785], (FIG. 3). Recently, it has been shown that patients with MC4R gene mutations (the receptor for α-MSH) have a strongly associated rate (100%) of Binge Eating Disorder, and are usually obese (Farooqi et al. supra; Branson et al. supra). They represent about 5% of the obese population (Farooqi et al, supra; Lustig supra). Patients with errors involving the cleavage of the POMC molecule, such that α-MSH is not produced, also have been noted to be obese, (FIG. 2), (Lustig supra). It would appear that there exists a neuroendocrine feedback loop which has been clearly illustrated in a recent editorial (FIG. 3), (List and Habener, supra), and that any function interruption of the feedback circuit appears to lead to obesity. Based on the above, it is predicted that the increased caloric intake of a patient on chronic steroids appears to be mediated via the decrease in α-MSH production that is associated with decreased hypothalamic and pituitary activity and ACTH and α-MSH production.

### SUMMARY OF THE INVENTION

The present invention relates to a method of treating patients with iatrogenically suppressed endogenous α-MSH production with exogenous α-MSH or an MC4R agonist. This treatment should result in a reduction in weight, and any ongoing weight gain, in those patients who are on chronic steroid therapy. Weight gain caused by binge eating should also be countered with this therapeutic approach. While patients undergoing chronic steroid therapy should be expected to have the best response to exogenous MC4R agonists, patients who are not on steroid therapy may also respond to this treatment. Also, patients treated with exogenous α-MSH agonists should have decreased leptin serum values. These leptin serum value alterations would be secondary to a feedback mechanism due to exogenous MC4R stimulation.

### DESCRIPTION OF THE DRAWINGS

FIG. 1 is a scheme illustrating that elevated leptin increases levels of TNF-a which reduces function of the insulin receptor and leads to the metabolic syndrome and also insulin resistant diabetes.

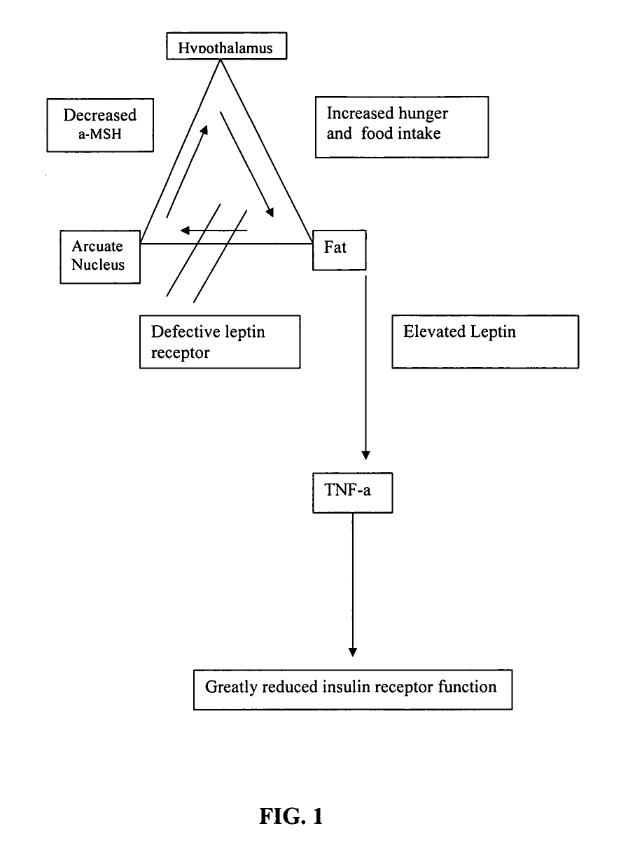

FIG. 2 is a scheme that illustrates that replacing the missing MC4R agonist will reduce patients' food intake.

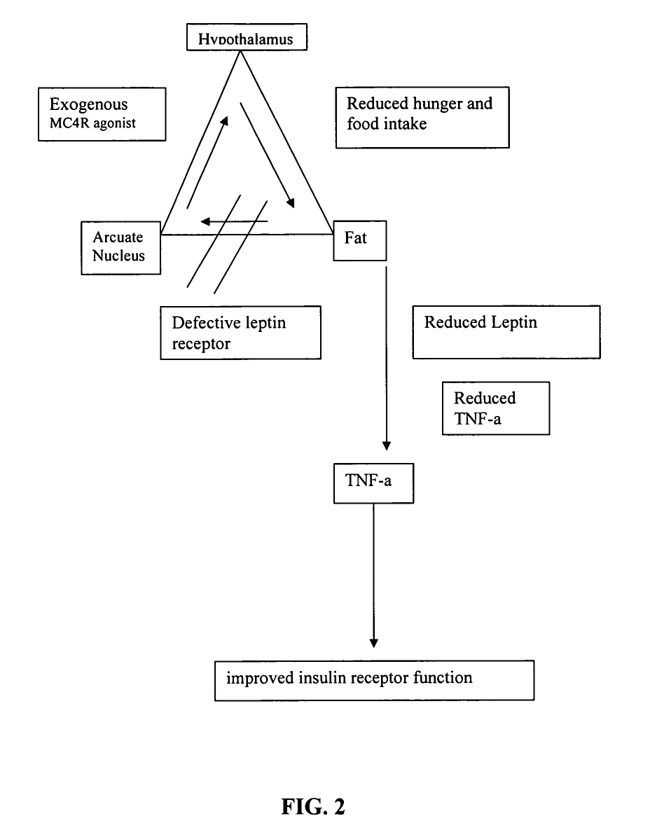

FIG. 3 is a schematic diagram showing a neuroendocrine feedback loop.

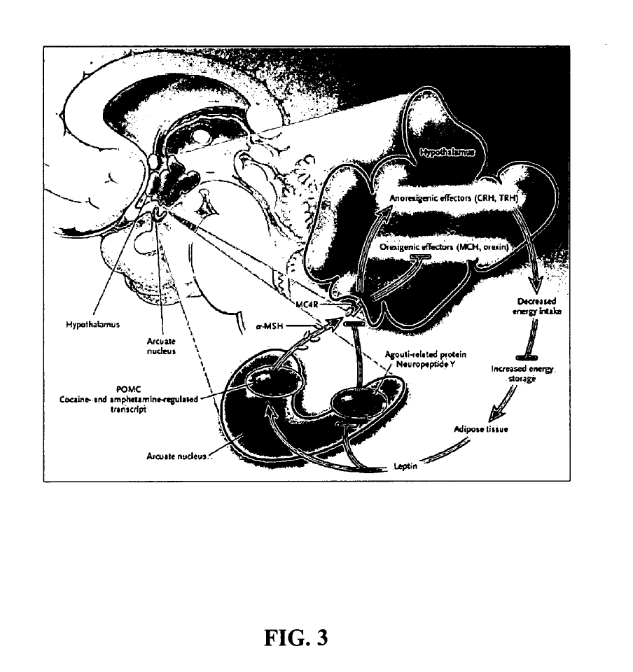

### DETAILED DISCUSSION OF THE INVENTION

In general the terms and phrases used herein have their art-recognized meaning, which can be found by reference to standard texts, journal references and contexts known to those skilled in the art.

The concepts and proposals discussed above come from a synthesis of recently published information relating to MC4R association with eating disorders, as well as an understanding of the neuroendocrine pathophysiological process associated with obesity. Upon review of the medial literature, a presumed pathway relating MC4R stimulation—or lack thereof—with weight changes is being hypothesized. The primary focus is on the lack of α-MSH in patients who are on chronic steroid therapy (and have low POMC and ACTH values). The natural inference, therefore, would be to use exogenous α-MSH, or any MC4R agonist, to correct this imbalance and presumably cause a weight loss in those who have chronically suppressed endogenous α-MSH levels. Interestingly, at this time, such synthetic hormonal drugs are in development and current clinical testing.

Dr. Handley, an endocrinologist at the University of Arizona (retired), has recently created a novel MC4R agonist named PT-141. This drug is being licensed to a company named Palatin Technologies. Currently they are using PT-141 for erectile dysfunction in patients who failed Viagra. They have completed phase I and phase IIA trials and are working now on phase IIB trials. Palatin reports that the drug was well tolerated and that the side effects observed were mild with no clinically significant adverse events noted (Competitive Technologies Press Release dated Jan. 1, 2003; Licensee Reports PT-141 Data for Males). Dr. Hadley's drug, PT-141 or Melanotan-II, a synthetic cyclic heptapeptide, Ac—Nle-c[Asp-His-DPhe-Arg-Trp-Lys]—NH₂, would be an ideal candidate for treating patients with steroid induced weight gain. However, any MC4R agonist would most likely be effective in treating weight gain in these patients. Indeed, Dr. Hanley stated that this group had not considered this possibility and felt that this idea held promise and merited further study. Upon pursuing the literature, this idea and concept have not been documented or reorganized as a viable treatment plan for those with obesity, particularly those who have suffered the sequelae of chronic steroid therapy. As such, the idea appears to be novel both in regard to the pathophysiological process as well as a possible treatment strategy.

One would also assume that leptin is associated either directly or indirectly with weight changes and that exogenous MC4R agonist therapy should decrease serum leptin levels. This would appear to be a natural consequence of stimulating the hypothalamic and pituitary neuroendocrine pathways that result in leptin generation peripherally.

Those skilled in the art will appreciate that the invention described herein is susceptible to variations and modifications other than those specifically described. it is to be understood that the invention includes all such variations and modifications. The invention also includes all of the steps, features, compositions and compounds referred to or indicated in this specification, individually or collectively, and any and all combinations of any two or more of said steps or features.

All references cited in the present application are incorporated in their entirety herein by reference to the extent not inconsistent herewith.

1. A method of decreasing steroid induced body weight gain, said method comprising administering an effective amount of an alpha-melanocyte stimulating hormone (α-MSH) agonist to a subject whereby a decrease in body weight gain in said subject is achieved.

2. The method of claim 1 wherein the α-MSH agonist is a synthetic molecule.

3. The method of claim 1 wherein the subject is a patient who is on chronic steroid therapy.
